(19) United States
(12) Patent Application Publication
(10) Pub. No.: US 2010/0049006 A1
(43) Pub. Date: Feb. 25, 2010

(54) MEDICAL SIGNAL PROCESSING SYSTEM WITH DISTRIBUTED WIRELESS SENSORS

(76) Inventors: Surendar Magar, Dublin, CA (US); Venkateswara Rao Sattiraju, Union City, CA (US)

Correspondence Address: WILSON, SONSINI, GOODRICH & ROSATI, 650 PAGE MILL ROAD, PALO ALTO, CA, 94304-1050, US

(21) Appl. No.: 12/096,195

(22) PCT Filed: Feb. 23, 2007
(86) PCT No.: PCT/US07/62772

§ 371 Date: Oct. 16, 2009

Related U.S. Application Data
(60) Provisional application No. 60/776,590, filed on Feb. 24, 2006.
(60) Provisional application No. 60/810,742, filed on Jun. 1, 2006.

(51) Int. Cl. A61B 5/00; A61B 5/0402

(52) U.S. Cl. 600/301; 600/509

(57) ABSTRACT

A wireless medical signal processing system for health monitoring is disclosed which achieves high wireless link reliability/security, low power dissipation, compactness, low cost and supports a variety of sensors for various physiological parameters. The system includes a medical signal processor which communicates with a wireless distributed sensor system as its peripheral for detecting physiological parameters of the person and for providing signals indicative thereof. The medical signal processor wirelessly receives the signals from the distributed wireless sensor system in a multiplexed fashion and processes the signals to provide an indication of the health of the person. The indication of health could relate to a disease state, general health or fitness level of a person. The system also includes a mobile device for receiving the indication of the health of the person to allow for a diagnosis or treatment of the person.

### CROSS-REFERENCE TO RELATED APPLICATIONS

Under 35 U.S.C. 119, this application is a Non-Provisional application of U.S. Provisional Application No. 60/776,590, filed Feb. 24, 2006 and U.S. Provisional Application No. 60/810,742, filed Jun. 1, 2006, all of which are incorporated herein by reference.

### FIELD OF THE INVENTION

The present invention relates generally for health monitoring and more particularly to a health monitoring system that utilizes a medical signal processor with a wireless distributed sensor system.

### BACKGROUND OF THE INVENTION

Monitoring the health of people has always been important. As the population ages and more people advance in age health monitoring systems become more significant to maintaining a healthy lifestyle and disease management. Remote health monitoring makes it easier and cost effective to monitor the health of vast populations. Wireless systems are the most desired approach to enable remote health monitoring. Therefore, a variety of wireless health monitoring systems have been introduced over the years.

Conventional wireless health monitoring systems are bulky, expensive, have inadequate wireless link reliability and have high power dissipation which severely limits their applications, particularly to monitor wide ranging physiological parameters in high volumes for large populations. Accordingly, what is desired is a system that addresses the above-identified issues.

### SUMMARY OF THE INVENTION

A wireless medical signal processing system for health monitoring is disclosed which achieves high wireless link reliability/security, low power dissipation, compactness, low cost and supports a variety of sensors for various physiological parameters. The system includes a medical signal processor which communicates with a wireless distributed sensor system as its peripheral for detecting physiological parameters of the person and for providing signals indicative thereof. The medical signal processor wirelessly receives the signals from the distributed wireless sensor system in a multiplexed fashion and processes the signals to provide an indication of the health of the person. The indication of health could relate to a disease state, general health or fitness level of a person. The system also includes a mobile device for receiving the indication of the health of the person to allow for a diagnosis or treatment of the person, and a secure server for securely storing the at least one indication of health. The core processing resources of the medical signal processor allows wireless distributed sensors to be ultra reliable/secure, ultra low power, ultra small and low cost. The peripheral wireless sensors can be a within a reasonable range of medical signal processor, such as within a typical home.

A distributed sensor based mobile/remote monitoring system for the management of various types of diseases is disclosed. The system is capable of continuously monitoring a variety of parameters relating to the state of various diseases. The parameter monitoring can be continuous, periodic or episodic. The system is capable of continuous monitoring of given parameters from a few seconds to many days. A system to manage a particular type of disease or meet a health objective can be defined by selecting the appropriate parameters for that disease.

### BRIEF DESCRIPTION OF DRAWINGS

FIG. 1A is a block diagram of a first embodiment of a general architecture of wireless health monitoring system in accordance with the present invention.

FIG. 1B is a block diagram of a second embodiment of a general architecture of a wireless health monitoring system in accordance with the present invention.

FIG. 2 illustrates examples of various sensors that can be included in a distributed sensor network.

FIG. 3 illustrates a block diagram of a wireless patch in accordance with the present invention.

FIG. 4 illustrates a block diagram of a medical signal processor in accordance with the present invention.

FIG. 5 is a block diagram of a cardiac care product in accordance with the present invention.

FIG. 6 is a block diagram of an implementation of a mobile device utilized with the cardiac care product of FIG. 5.

### DETAILED DESCRIPTION

The present invention relates generally to health monitoring and more particularly to a health monitoring system that utilizes a medical signal processor with a wireless distributed sensor system. The following description is presented to enable one of ordinary skill in the art to make and use the invention and is provided in the context of a patent application and its requirements. Various modifications to the preferred embodiments and the generic principles and features described herein will be readily apparent to those skilled in the art. Thus, the present invention is not intended to be limited to the embodiments shown, but is to be accorded the widest scope consistent with the principles and features described herein.

To describe the feature of the medical signal processing system in more detail, refer now to the following description in conjunction with the accompanying figures.

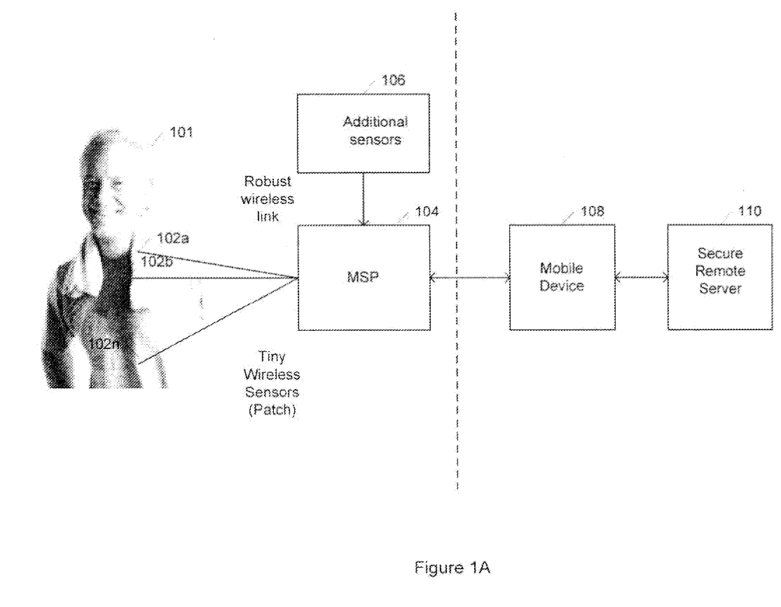

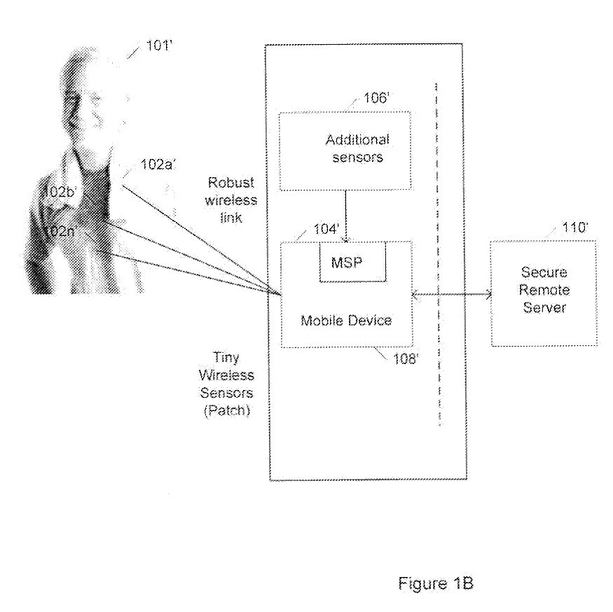

FIG. 1A is a first embodiment of a general architecture of a wireless medical signal processing system 100 in accordance with the present invention. The system 100 is centered around a medical signal processor 104 that has a wireless distributed sensor network as its peripheral. The distributed sensor network includes a plurality of patches 102a-102n on a person 101. The patches 102a-102n can be internal to the body, coupled to the exterior of the body embedded in the garments or can be in close proximity of the body by some other means. The patches communicate wirelessly with MSP 104. The MSP 104 also includes its internal/local sensors 106, which can engage the body of the person, which are also part of the distributed sensor system. The medical signal processor (MSP) 104 in turn communicates with a mobile device 108. The mobile device 108 in turn communicates with a secure server 110 via a wireless or wired network. In this embodiment, the MSP 104 is a separate component from the mobile device 108. However, one of ordinary skill in the art readily recognizes that the MSP 104 could be incorporated into the mobile device as shown in FIG. 1B which is a second embodiment of the system 100′. The MSP 104 also includes sensors 106, which can engage the body of the person, which are also part of the distributed sensor network. The MSP 104 has the ability to absorb significant processing burden from all of its distributed sensors to form a reliable wireless link with them. The MSP 104 also has the ability to communicate with all of its distributed sensors through a wireless uplink. It allows the MSP 104 to use its internal resources to monitor, control and dictate various performance factors of the distributed sensors to achieve the performance balance needed for any given application. The MSP 104 also can perform various house keeping functions for the overall medical signal processing system.

The mobile device 108 could be, for example, a cellular telephone, laptop, notebook, a smart phone, a PDA, a custom medical device or any mobile device which can communicate with the server over a network. Each component of the health monitoring system 100 will now be described in detail in conjunction with the accompanying figures.

### Medical Signal Processing System

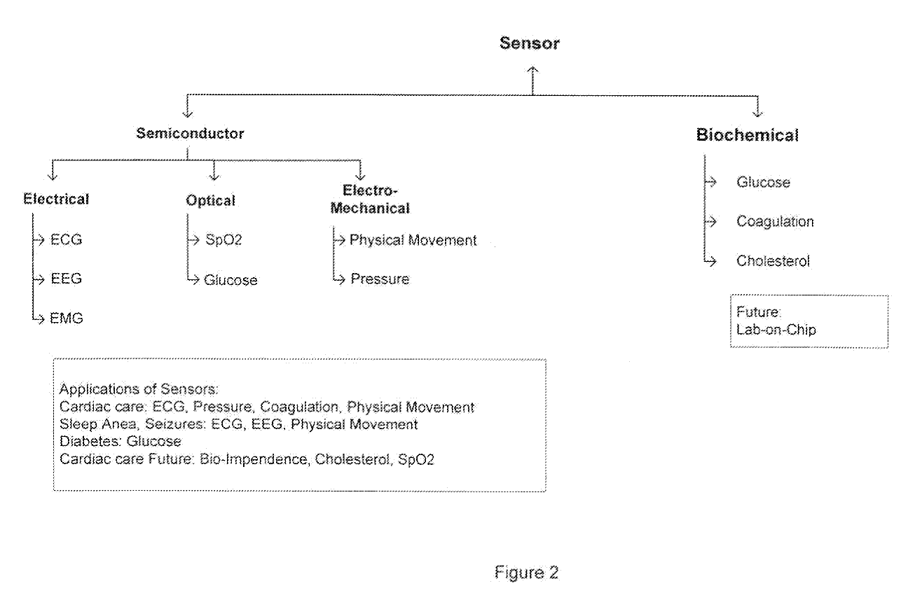

As discussed above, the medical signal processing system as shown in FIGS. 1A and 1B can include a variety of sensors—either directly integrated in the medical signal processor 104, or linked to the medical signal processor 104 via a wireless link as patches 102 on the body of a user. Examples of various sensors that can be included in the distributed sensor system are shown in FIG. 2. Out of these examples, certain sensors can be chosen for implementation as patches 102. Other sensors can be chosen for integration within the MSP 104. In this way, a variety of systems can be designed for the management of diseases, health and fitness, by choosing the sensors that monitor the appropriate parameters associated with target applications.

Modes of Operation: By using the distributed sensor network, the system of FIGS. 1A and 1B can monitor parameters in different ways. For example, by wearing patches on the body, the monitoring can be done continuously—continuously data flowing from sensors in to the mobile device to the secure server. Patches can also be used for periodic or episodic monitoring. In a stand-alone mode, monitoring is normally done in an episodic or periodic mode by using the MSP 104 and sensors 106. For example, a cardiac rhythm can be directly monitored by pressing the MSP 104 against the body by using a built in ECG sensor. Another example of this stand-alone mode is glucose, cholesterol or blood coagulation monitoring. A drop of blood can be placed on a biochemical sensor that is built into the MSP 104 which can be converted to electrical signal by MSP for further processing. The glucose, cholesterol or blood coagulation rate reading will be registered in the database on MSP 104 and/or mobile device 108 and/or the secure server 108.

### Wearable Wireless Patches 102

Patches 102 are integrated circuit technology driven miniature wireless devices that can be conveniently attached to the body. Patches can also be designed for implanting within the body of a person. To achieve compactness, the patches 102 are designed using custom ASIC and a compact multi-chip module. The patches can be further simplified by leveraging the resources of MSP 104. The patch 102 in a preferred embodiment has two main parts: sensor circuits, and a wireless radio core for the transmission of sensor data to other devices. In addition, it has a signal processor and power management circuits to achieve very low power dissipation. The sensor circuits can be directly incorporated in the custom ASIC and/or patch can also include a stand-alone sensor device whose data can be transmitted to other devices using the wireless radio or ASIC on the patch. In a preferred embodiment, a person can wear a patch 102 for several days for continuous monitoring without changing or recharging the power source. Patches 102 can have the ability to receive wireless signals from the MSP 104 to enhance its own power dissipation and improve its own wireless link reliability, based on the MSP's 104 monitoring of radio environment and application requirements. The patches 102 can also receive test/control signals from the MSP 104 to get authenticated and to check its own functionality.

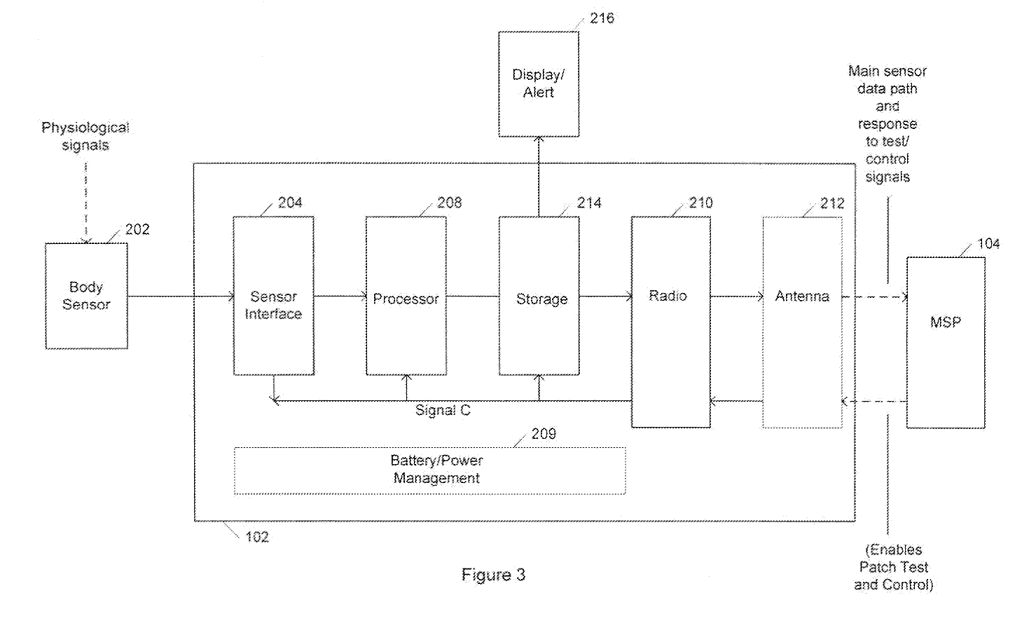

FIG. 3 illustrates a wireless patch 102 utilized in accordance with the present invention. The wireless patch 102 receives signals from a body sensor 202 via a sensor interface 204. The patch 102 may receive signals from a body it is either in contact with, or in close proximity of. The sensor interface 204 can receive electrical signals or other signals representative of different physiological parameters of the body. The output from the sensor interface 204 is provided to a processor 208 which processes the signal to perform various functions such as compression to reduce the data rate, encoding to achieve high reliability and manage buffering to vary duty cycle of radio. The processed data is presented to a storage element 214. The data from the storage element 214 is provided to a radio 210 which outputs the signal to a signal antenna 212. The storage element 214 can be adapted to be coupled to a local display/alert 216. A power source 209 provides power and power management to all elements of the patch 102. As shown, a wireless path through radio/antenna 210/212 also exists to receive test and control signals from the MSP 104 as discussed above. As shown, all resources of the patch 102 can be controlled by the MSP 104 by a signal C, wirelessly coming to patch 102 from the MSP 104.

Accordingly, by leveraging the information sent by MSP 104 via signal C, patches can dynamically alter the performance of their various functional blocks to choose trade off among high reliability, high security, low power and low cost for given applications of health monitoring.

In summary, the trade off is possible due to any of or any combination of the following features:
a. A sensor interface to connect to a variety of physiological sensors
b. A radio subsystem that can support a variety of communication schemes (e.g. different modulations including analog modulation, various codings, various data rates) to wirelessly communicate with a medical signal processor which is within a reasonable range, such as within a typical house
c. A processor to support a variety of wireless communication schemes for radio system
d. A processor that can implement various authentication and security schemes as desired by application
e. Means to wirelessly receive a variety of test signals from a medical signal processor
f. Means to run test signal though its data paths and generate output signals in response
g. Means to wirelessly send resulting output signals back to medical signal processor
h. Means to receive various control signals to reconfigure its various functional blocks
i. Reconfigurable internal blocks to alter data rates, radio scheme, communication algorithm, power dissipation levels, etc.
j. sensors that can receive body's electrical physiological signals
k. Encapsulation in a packaging material that can also provide a body interface
l. Using its radio, generation of a RF beam that can be directed towards a part of person's body to probe internal parts
m. Means to receive the RF signals scattered by body that can be analyzed to get information about the internals of the body
n. Means to bring the device in a close proximity of body
o. Means to attach the device to body
p. Means to analyze and display the sensor data
q. Means to alert a person as needed
r. For ultra high reliability, ability of patches to wirelessly communicate with each other in case of loss of link by a patch to medical signal processor

### Medical Signal Processor (MSP) 104

The medical signal processor (MSP) 104 collects and receives data from the one or more of the distributed sensors (internal or external), and aggregates and processes this data. In addition, the MSP 104 can reliably transmit it to mobile device 100 in such a way that mobile device 100 in turn can transmit the data to a remote server system over wireless, cellular, or any type of wide area network (WAN).

The MSP 104 may have one or more of the following features:
1. to collect data from its internal/local sensors
2. radio/processors to receive data from external wireless sensors that are within a reasonable range, such as within a typical house
3. means to process and aggregate the sensor data based on an algorithm that can be programmed in MSP 104 to determine a diseases state and/or health state and/or fitness state
4. means to attach or connect or plug in to a mobile device
5. means to generate an alert based on the determination of the state of disease, health or fitness
6. means to locally display collected raw sensor data or processed data
7. means for transmission of collected raw sensor data or processed data to a remote server either directly or via a mobile device
8. means to enable continuous reliable transmission of sensor data over a cellular or wide area network
9. user interface to control the operation of monitoring system
10. means of a regular cell phone device (voice, data and image communication, display, keypad, etc.)

In addition to collecting and processing the data from all of its peripheral patches/sensors, the MSP 104 also has various means to wirelessly monitor and control all of its peripheral patches/sensors through a wireless uplink with them. Essentially, the MSP 104 becomes an integral part of the wireless medical signal processing system to achieve the overall requirements of the system—a major requirement being patches to be ultra reliable/secure, ultra low power, ultra small and low cost. The overall functionality of the system is asymmetrically partitioned between the patches 102 and MSP 104 to achieve these critical patch requirements.

Accordingly, MSP 104 may have the following features to achieve the system objectives:
1. means to act as a master of the overall system and patches/sensors to be its slaves
2. means to manage a distributed network of patches/sensors
3. means to authenticate, test and control the functionality of all of its peripheral patches/sensors
4. means to monitor/dictate the wireless link performance of its peripheral patches/sensors
5. means to monitor/dictate the power dissipation of its peripheral patches/sensors
6. means to dictate the degree of reliability of all of its peripheral sensors/patches
7. means to allow peripheral sensors/patches to use a very simple radio and have its own signal processor to complete the radio processing for patches/sensors
8. means to recreate the original sensor data if data has been compressed on the sensor/patches
9. means to monitor radio environment.
10. Radio to work with multiple communication schemes including digital and analog modulation

The MSP 104 can control the functionality and performance of its peripheral/patches based on the requirement defined for the overall system. The system performance can be dynamically adjusted, for example, due to a change in radio environment or a change in person's condition as monitored by the MSP 104.

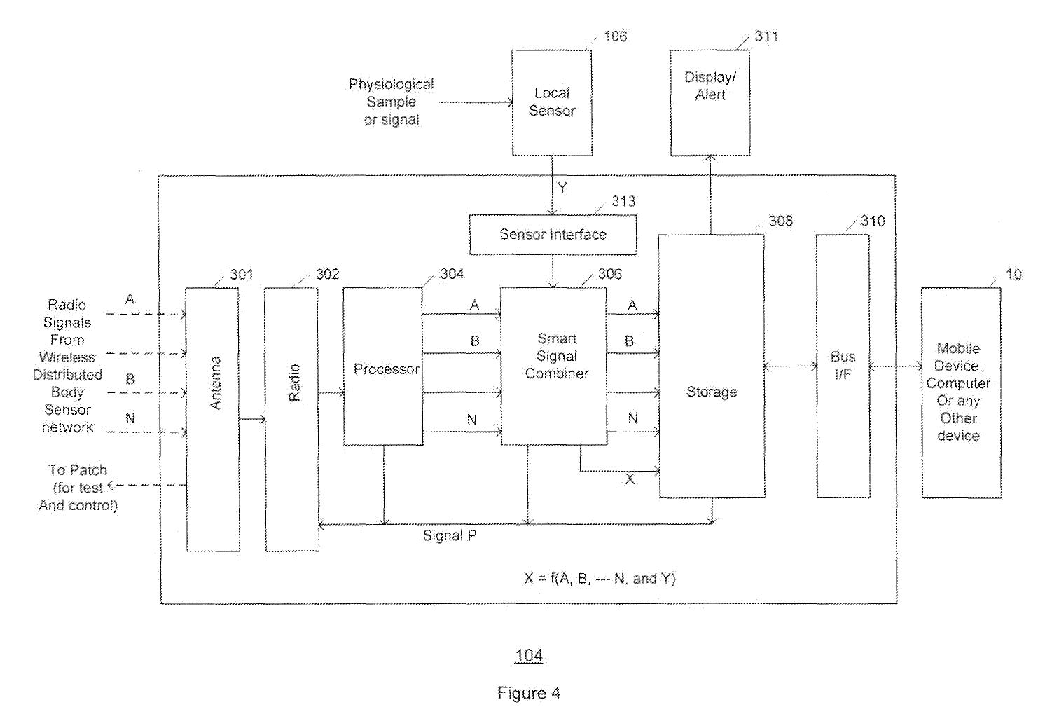

FIG. 4 illustrates an MSP 104 in accordance with the present invention. An antenna 301 and a radio 302 within the MSP 104 receives a plurality of data signals (signals A-N) from the distributed sensor network. The radio 302 then provides these signals to a signal processor 304. The processor 304 then decodes the signals received by the radio 302. The decoded signals are then provided to a smart signal combiner 306, in a multiplexed or parallel fashion.

The smart signal combiner 306 includes a means for programming an algorithm for combining the signals to provide an indication of a state of the body. For example, certain sensor parameters taken together might indicate a disease state and/or heath state and/or fitness state of an individual.

The smart signal combiner 306 may also receive a signal Y from the local sensors 106′ in the MSP 104. The signal Y represents either one signal from one local sensor or a plurality of signals from a plurality of local sensors. The smart signal combiner 306 also provides a signal (X) that is a parameter, relating to a state that has been measured utilizing a single sensor output or by combining the outputs of multiple sensors. This state is a result of one or several physiological parameters of the body and the signal X may be a function, computed over time, of one, all or a set of those sensor outputs (signals A-N) and sensor signals.

These various signals (A, B, . . . N, Y, X) are provided to a storage element 308 by the smart signal combiner 306. The storage element 308 may be any type of memory that can be utilized in integrated circuits. The storage element 308 can be adapted to be coupled to a local display/alert device 311 via the sensor interface 313. The data can then be retrieved by the mobile device from the storage element 308 via a bus interface 310. As before mentioned, the MSP 104 can either be part of the mobile device 108 or a stand alone device.

All these resources enable MSP 104 to act as a stand-alone device to provide the needed information locally to concerned parties or it can transmit the information to a remote secure server for further processing and access. The information can be used locally, or remotely, to diagnose/treat a disease or for general health/fitness management of a person. As shown, MSP 104 also has a wireless path to communicate with patches/sensors to monitor and control their performance. In a control mode, radio 302 operates in an uplink mode by sending test/control data via signal P over the wireless link. This control mode is activated when the MSP 104 needs to test, monitor and/or control its peripheral patches/sensors via the processor 304. The processor 304 should be for example, a microprocessor with signal processing capability that executes the various functions.

The processor 304 can utilize other resources such as smart signal processor 306 and storage 308 to carry out its control/test related and general processing tasks. In the control mode, for example, the processor 304 can generate test signals and send to a patch 102, and analyze the signals received from the patch 102 to estimate its wireless link performance. If needed, the MSP 104 can then send control signals to alter the wireless link performance by changing certain parameters relating to radio functions of the patch 102, for example by instructing signal processor 208 and radio 210. In some implementations, some of the internal blocks of MSP 104, such as processor 304, smart signal processor 306 and storage 308 can be implemented in software. This implementation is likely when MSP 104 functionality is embodied within a mobile device, computer, a custom medical device, or any other device.

The functionality of MSP 104 allows its distributed sensors (patches) to maintain high wireless reliability, high security, low power and low cost. Furthermore, the versatility of MSP 104 allows it to create a variety of different types of medical systems. To allow this functionality and versatility, in summary, it can include any of or any combination of the following features:
a. Means to wirelessly communicate with a plurality of peripheral wireless physiological sensors in a multiplexed fashion that are within a reasonable range, such as within a typical house
b. Means to manage a network of plurality of said sensors as their master
c. Means to display health state information or the data received from peripheral sensors
d. Means to alert a person about health state
e. Means to connect to a mobile device to exchange information with it and to communicate with a remote server through mobile device's connectivity to a wide area network
f. Partitioning of its functions between hardware and software to allow its integration within a mobile device
g. Means to wirelessly send a variety of test signals to its peripheral sensors and analyze the received signals to monitor the proper functioning of the sensors and their various internal functional blocks
h. Means to send various control signals to peripheral sensors to configure their various functional blocks:
i. Means to monitor peripheral sensors to determine their respective power dissipation rates and the state of their power sources; to supervise power management
j. Means to monitor surrounding radio environment to determine an optimum wireless communication scheme at any given instance
k. Means to instruct peripheral sensors to utilize a particular radio/communication mode for reliable operation
l. Means to authenticate peripheral sensors
m. Means to monitor various security aspects of peripheral sensors
n. Means to allow coupling to local sensors through a wired connection
o. A processor to support the execution of a variety of communication algorithms/schemes to allow peripheral sensor to use the simplest possible communication scheme for a given application to minimize sensor's power and resource requirements by absorbing the burden of processing (asymmetric communication scheme)
p. A smart signal combiner that can be programmed to run needed algorithms to (i) analyze signals from one or more peripheral sensors over time, and/or (ii) combine signals from a plurality of peripheral sensors; to determine a health state
q. Storage media to store the health state information and/or raw data received from peripheral sensors

### Mobile Device 108

The mobile device 108 could be, for example, a cellular telephone, laptop, notebook, a smart phone, a PDA, a custom medical device or any mobile device which can communicate with the server over a wide area network and/or Internet. The mobile device 108 can also be a regular cell phone handset, which has been modified to include the appropriate features and means to work with MSP 104. The mobile device 108 communicates with the MSP 104. In one embodiment, the MSP can be built within mobile device 108 as part of the mobile device design. In this mode, many internal functions of MSP can be implemented in software. In most cases, MSP's radio system and sensor interfaces will remain intact in hardware.

### Secure Server 110

The secure server 110 receives data from distributed sensors over a cellular telephony network, any type of wide area network or Internet via MSP 104 and the mobile device 108. The server 110 further processes the received data from the mobile device and stores it in a secure location. The server 110 may also contain various types of software programs, including software to manage health information databases (such as electronic medical records, computerized purchase orders and computerized prescription systems). The secure server 110 may also have the middleware to process/link sensor data to such health information databases.

The data stored on the secure server 110 may be accessed by a healthcare provider, caregiver or patient via the Internet by using any type of terminal device such as computer, mobile device, cell phone, smart phone or personal data assistant (PDA).

The health monitoring system in accordance with the present invention supports many classes of sensors for physiological data collection, such as:

1. The health monitoring system supports many classes of sensors for physiological data collection, such as:
(a) Sensors (either patches 102 or sensors 106) contacting the body 101 through gels, etc.
(b) Patches 102 embedded within the body 101 through surgical procedures.
(c) Patches 102 probing the body 101 through micro-needle based skin punctures.
(d) Sensors in close proximity of the body 101—e.g., probing using a microwave or optical beam.
(e) Sensors embedded in the MSP 104 or mobile device 108 for periodic or occasional use.
(f) Sensors that can read biochemical micro-fluidic test strips (e.g. glucose, blood coagulation rate) via electrical or optical sensor

2. The health monitoring system in accordance with the present invention can support one of these sensors and/or patches or multiple sensors and/or patches from multiple classes.

3. The MSP 104 has the ability to collect data in real time from many such sensors and/or patches and to apply a chosen algorithm to combine signals from various sensors and/or patches to determine or predict a physiological or disease state.

4. The MSP 104 can store data for local use and/or transmit in real time to a remote server for use by clinicians and other parties. If desired, some of the MSP 104 functions can be implemented on a remote sensor.

5. As stated above, one function of the MSP 104 is physiological data processing.

6. The second function of MSP 104 is to manage all patches and/or sensors for optimal functionality—managing authentication/security functions, monitor and enhance the radio transmission performance of patches and/or sensors to increase link reliability, monitor and minimize power dissipation by patches and/or sensors.

The health monitoring system in accordance with the present invention can be utilized in a variety of environments. One example is the cardiac disease management system. To describe the features of such a system refer now to the following description in conjunction with the accompanying figures.

### A Mobile/Remote Monitoring System for Cardiac Disease Management

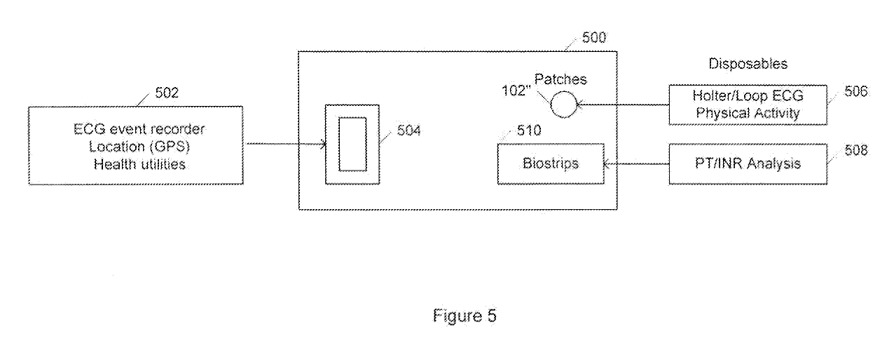

An embodiment of a cardiac disease care product in accordance with the present invention is described herein below. FIG. 5 is a block diagram of a cardiac care product in accordance with the present invention. The cardiac care product includes a mobile device 504 which utilizes patches 102″ and biostrips as sensors 510. The mobile device 504 includes an ECG event recorder 502, a geographic positioning system (GPS) and health utilities. The patches may include a holter mechanism and a loop ECG monitor 506 as well as accelerometers for detecting physical activity. The biostrips 510, which are basically microfluidic test strips, may be utilized, for example, for anticoagulation analysis of the blood (PT/INR).

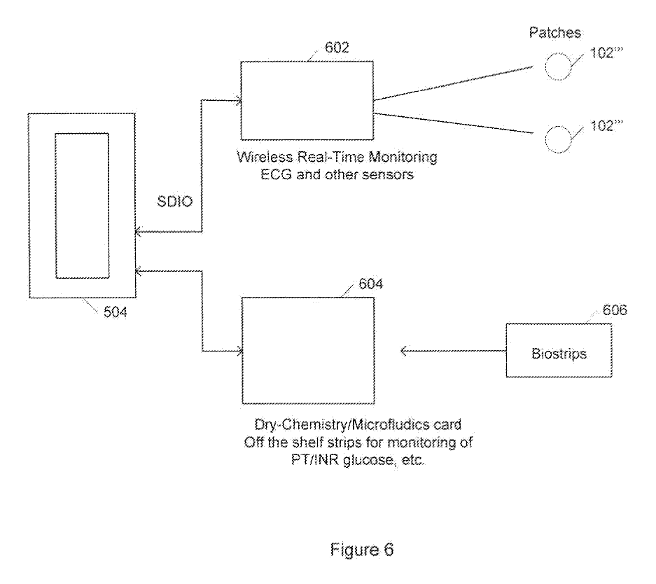

FIG. 6 is a block diagram of one implementation of a mobile device utilized with the cardiac care product of FIG. 5. The mobile device 504 may receive a first SD (Secure Digital) card 602 that includes wireless real-time monitoring system. The SD card 602 receives data from patches 102″″ and other sensors. The mobile device 504 also may receive a second SD card 604 that monitors PT/INR, glucose and the like. The second SD card receives its data via biostrips 606 that can be activated by a drop of patient's blood, for example. The PT/INR and/or glucose reading is obtained by building an electrical or optical reader on the second SD card that can read the biostrips. The cardiac care product can be used for the management of various cardiac diseases, including arrhythmia. In an embodiment, this cardiac care product monitors the following parameters:
1. ECG signals (time duration programmable—few seconds to few weeks)
2. Pulse and respiration
3. Patient's physical movement
4. Blood coagulation analysis for drug therapy for the treatment of arrhythmia
5. A mobile, integrated system for remote cardiac care is provided—It is a system that is useful for diagnosis and treatment of various cardiac diseases.
6. Its core functions are listed below:
   (a) Wireless AECG—duration programmable from a few seconds to 30 days to serve a variety of functions including holter monitoring, cardiac event monitoring, cardiac loop monitoring (wireless ECG sensor patches and receiver)
   (b) PT/INR based blood anticoagulation analysis (dry chemistry microfluidic strip and optical/electrical reader/sensor)
7. Its auxiliary functions are listed below:
   (a).Patient activity recording (accelerometer sensor)
   (b).Patient location information (GPS)
   (c).Ability to connect to an implanted wireless pacemaker
   (d).Medication schedules (software)
   (e).Doctor visit and treatment schedule (software)
8. Microfluidic biostrip/reader concept can also be used for glucose monitoring
9. The system can be built by integrating the electronics inside a mobile device/computer or an attachment to a mobile device/computer. The mobile device 504 may or may not be connected to a remote server through a network.

The sensors for parameter monitoring may be distributed between the patches 102″″ and the mobile device 504 as follows:

### Patches 102

The patches 102″″ have sensors to continuously monitor ECG, pulse, respiration and patient's physical movement. ECG function can be programmed to work in any mode as prescribed by a physician, such as:
(a) Continuous ECG: for any amount of time (e.g. 24 Hrs, 48 Hrs, seven days, thirty days).
(b) ECG Loop recorder: Shorter time recordings with continuous overwriting

Patient's physical movement data is recorded along with ECG data on a continuous basis. In addition, pulse and respiration are recorded as desired.

### MSP 104

In a stand-alone mode, the mobile device 504 has the means to monitor a few different parameters as below:
(a) ECG Event Recording: Via built-in ECG sensor, mobile device 504 is able to record ECG signals for any duration as desired. In this mode, the mobile device 504 is directly held to the body 101.
(b) Biochemical parameters: The mobile device 504 has a built in biochemical sensor, electrical sensor and an optical sensor. Any of these sensors can be used to read certain parameters relating to disease management. For example, the MSP 104 can register blood coagulation readings for PT/INR (Prothrombin Time/International Test Ratio) analysis for Warfarin drug therapy. For this application, a test strip with a blood drop mixed with a chemical reagent can be inserted into the MSP 104 to determine blood anticoagulation rate for PT/INR analysis.

### CONCLUSION

A distributed sensor based mobile/remote monitoring system for the management of various types of diseases is disclosed. The system is capable of continuously monitoring a variety of parameters relating to the state of various diseases. The parameter monitoring can be continuous, periodic or episodic. Some of the parameters that can be monitored by the system are ECG (electrocardiograph), EEG (electroencephalograph), EMG (Electromyography), blood glucose, pulse, respiration, blood pressure, temperature, SpO2, body fluid density, blood density, patient physical movement and patient physical location. A system to manage a particular type of disease can be defined by selecting the appropriate parameters for that disease. The system can be applied to manage many type of diseases and conditions, such as—arrhythmia, heart failure, coronary heart disease, diabetes, sleep apnea, seizures, asthma, COPD (Chronic Obstructive Pulmonary Disease), pregnancy complications, wound state, etc.

An innovative technology base is needed to address wide ranging applications and to meet critical requirements for the mass market—high reliability, high security, low power, small form factor and low cost. The technology disclosed meets this goal. The technology involves a medical signal processor (MSP) closely supervising all aspects of functionality of its peripheral wireless patches to help achieve the objectives. The patches are simple while the medical signal processor (MSP) has all the smarts to work with patches. It results in asymmetric processing load on MSP and patches—patches are simple and reconfigurable and MSP has the complexity to take the processing burden from them for wireless communication link, and processing load to supervise patches. Both the MSP and the patches have various resources to build complete self contained systems to determine a health state of a person from sensor physiological data and to display and/or send data to another device for further processing.

Although the present invention has been described in accordance with the embodiments shown, one of ordinary skill in the art will readily recognize that there could be variations to the embodiments and those variations would be within the spirit and scope of the present invention. Accordingly, many modifications may be made by one of ordinary skill in the art without departing from the spirit and scope of the appended claims.

1.-65. (canceled)

66. A system for wireless healthcare monitoring comprising:
(a) a physiological signal monitoring patch comprising a sensor interface that receives signals from sensors coupled to the sensor interface, a signal processor coupled to the sensor interface, a storage element coupled to the signal processor, a radio coupled to the storage element, an antenna coupled to the radio, power dissipation management circuits coupled to the processor, and a battery that provides power to the patch; and
(b) a medical signal processor comprising an antenna that receives data from the patch, a radio coupled to the antenna, a storage element coupled to the radio, a processor coupled to the storage element, power dissipation management circuits, and a bus interface that communicates with a device.

67. The system of claim 66 wherein the medical signal processor is incorporated into the device.

68. The system of claim 66 wherein the medical signal processor is a stand-alone unit that is connected to the device.

69. The system of claim 67 wherein the device is a mobile device, smart phone, personal digital assistant, or medical device.

70. The system of claim 66 wherein the patch comprises a custom ASIC wherein the custom ASIC comprises the sensor interface.

71. The system of claim 66 wherein the processor in the patch performs compression to reduce data rate, performs encoding to achieve high reliability, and/or manages buffering to vary the duty cycle of the radio in the patch.

72. The system of claim 66 wherein the medical signal processor decodes the signal received from the patch.

73. The system of claim 66 wherein the medical signal processor authenticates, tests, and/or controls the functionality of one or more patches in the system.

74. The system of claim 66 wherein the medical signal processor dictates the power dissipation of the patches.

75. The system of claim 66 wherein the medical signal processor sends control signals to alter wireless link performance by changing parameters relating to radio functions by instructing the signal processor and/or the radio on the patch.

76. The system of claim 66 wherein the medical signal processor instructs patches to utilize a particular radio or communication scheme.

77. The system of claim 66 wherein the system further comprises a secure server with which the device communicates.

78. The system of claim 77 wherein the secure server links the sensor data with other health information.

79. The system of claim 66 wherein the physiological signals are monitored continuously, periodically, or episodically.

80. A system for wireless healthcare monitoring comprising:
(a) a physiological signal monitoring patch comprising a sensor interface that receives signals from sensors coupled to the patch, a signal processor coupled to the sensor interface, a storage element coupled to the signal processor, a radio coupled to the storage element, an antenna coupled to the radio, power dissipation management circuits coupled to the processor, and a battery that provides power to the patch; and
(b) a medical signal processor comprising an antenna that receives data from the patch, a radio coupled to the antenna, a storage element coupled to the radio, a processor coupled to the storage element, a storage element for storing data relating to the signals, power dissipation management circuits, and a signal combiner that decodes signals received from the processor to provide an indication of a state of a body.

81. The system of claim 80 wherein the signal combiner also receives signals from at least one local sensor connected to the medical signal processor.

82. The system of claim 80 wherein multiple sensor parameters taken together indicate a disease state, heath state, and/or fitness state of an individual.

83. The system of claim 66 or 80 wherein the system monitors ECG (electrocardiograph), EEG (electroencephalograph), EMG (Electromyography), blood glucose, pulse, respiration, blood pressure, temperature, SpO2, body fluid density, blood density, patient physical movement, patient physical location, or a combination thereof.

84. The system of claim 66 or 80 wherein the system monitors arrhythmia, heart failure, coronary heart disease, diabetes, sleep apnea, seizures, asthma, COPD (Chronic Obstructive Pulmonary Disease), pregnancy complications, wound state, or a combination thereof.

85. A patch for monitoring physiological signals comprising a sensor interface that receives signals from one or more sensors coupled to the sensor interface, a signal processor coupled to the sensor interface, a storage element coupled to the signal processor, a radio coupled to the storage element, an antenna coupled to the radio that sends and/or receives wireless signals, power dissipation management circuits coupled to the processor, and a battery that provides power to the patch; wherein the patch comprises a custom ASIC wherein the custom ASIC comprises the sensor interface, the signal processor, and the radio.

86. The patch of claim 85 wherein the physiological signals are ECG signals.

87. The patch of claim 86 wherein the patch measures ECG for 24 hours or more.

88. The patch of claim 66 wherein the patch monitors ECG (electrocardiograph), EEG (electroencephalograph), EMG (Electromyography), blood glucose, pulse, respiration, blood pressure, temperature, SpO2, body fluid density, blood density, patient physical movement, patient physical location or a combination thereof.

89. The patch of claim 85 wherein the patch monitors arrhythmia, heart failure, coronary heart disease, diabetes, sleep apnea, seizures, asthma, COPD (Chronic Obstructive Pulmonary Disease), pregnancy complications, wound state, or a combination thereof.

90. A patch for monitoring physiological signals comprising a sensor interface that receives signals from sensors coupled to the sensor interface, a signal processor coupled to the sensor interface, a storage element coupled to the signal processor, a radio coupled to the storage element, an antenna coupled to the radio that communicates wireless signals, power dissipation management circuits coupled to the processor, and a battery that provides power to the patch; wherein the patch is designed to be controlled by wirelessly communicated instructions.

91. The patch of claim 90 wherein the patch measures ECG, EMG, EEG signals, or a combination thereof.

92. A method for wireless healthcare monitoring of an individual comprising:
(a) sending a data signal from a physiological signal monitoring patch; wherein the patch comprises a sensor interface that receives physiological signals from sensors coupled to the sensor interface, a signal processor coupled to the sensor interface, a storage element coupled to the signal processor, a radio coupled to the storage element, an antenna coupled to the radio;
(b) managing power dissipation in the patch with management circuits on the patch coupled to the processor, wherein the patch comprises a battery that provides power to the patch;
(c) receiving the data signal at a medical signal processor comprising an antenna that receives the data signal from the patch, a radio coupled to the antenna, a storage element coupled to the radio, a processor coupled to the storage element, and power dissipation management circuits, and
(d) sending processed data from the medical signal processor to a device through a bus interface.

93. The method of claim 92 wherein the medical signal processor is incorporated into the device.

94. The method of claim 92 wherein the medical signal processor is a stand-alone device that is connected to the device.

95. The method of claim 93 wherein the device is a mobile device, smart phone, personal digital assistant, or medical device.

96. The method of claim 92 wherein the patch comprises a custom ASIC wherein the custom ASIC comprises the sensor interface.

97. The method of claim 92 wherein the processor in the patch performs compression to reduce data rate, performs encoding to achieve high reliability, and/or manages buffering to vary the duty cycle of the radio in the patch.

98. The method of claim 92 wherein the medical signal processor decodes the signal received from the patch.

99. The method of claim 92 wherein the medical signal processor authenticates, tests, and/or controls the functionality of the patch.

100. The method of claim 92 wherein the medical signal processor dictates the power dissipation of the patches.

101. The method of claim 92 wherein the medical signal processor sends control signals to alter the wireless link performance by changing certain parameters relating to radio functions by instructing the signal processor and/or the radio on the patch.

102. The method of claim 92 wherein the medical signal processor instructs patches to utilize a particular radio or communication scheme.

103. A method for wireless healthcare monitoring comprising:
(a) sending data from a physiological signal monitoring patch comprising a sensor interface that receives signals from sensors coupled to the patch, a signal processor coupled to the sensor interface, a storage element coupled to the signal processor, a radio coupled to the storage element, an antenna coupled to the radio;
(b) managing power dissipation on the patch with power management circuits coupled to the processor, wherein the patch comprises a battery that provides power to the patch;
(c) receiving the data at a medical signal processor comprising an antenna that receives data from the patch, a radio coupled to the antenna, a storage element coupled to the radio, a processor coupled to the storage element, a storage element for storing data relating to the signals, power dissipation management circuits; and
(d) combining the data on a signal combiner that decodes signals received from the processor to provide an indication of a state of a body.

104. The method of claim 103 wherein the signal combiner also receives signals from at least one local sensor connected to the medical signal processor.

105. The method of claim 103 wherein certain sensor parameters taken together indicate a disease state, heath state, and/or fitness state of an individual.

106. The method of claim 92 or 103 wherein physiological signal is related to ECG (electrocardiograph), EEG (electroencephalograph), EMG (Electromyography), blood glucose, pulse, respiration, blood pressure, temperature, SpO2, body fluid density, blood density, patient physical movement or patient physical location or a combination thereof.

107. The method of claim 92 or 103 wherein the method is used to monitor arrhythmia, heart failure, coronary heart disease, diabetes, sleep apnea, seizures, asthma, COPD (Chronic Obstructive Pulmonary Disease), pregnancy complications, wound state, or a combination thereof.
